(19) United States
(12) Patent Application Publication
(10) Pub. No.: US 2009/0069849 A1
(43) Pub. Date: Mar. 12, 2009

(54) DYNAMIC SCREW SYSTEM

(76) Inventors: YoungHoon Oh, Montville, NJ (US); Mahmoud F. Abdelgany, Rockaway, NJ (US)

Correspondence Address: Rahman LLC, 10025 Governor Warfield Parkway, Suite 110, Columbia, MD, 21044, US

(21) Appl. No.: 11/852,360

(22) Filed: Sep. 10, 2007

(51) Int. Cl. A61B 17/70; A61B 17/56; A61B 17/04; A61B 17/58

(52) U.S. Cl. 606/246; 606/100; 606/278; 606/301; 606/305; 606/308

(57) ABSTRACT

A dynamic screw system for stabilizing a vertebral body includes a bone screw adapted to connect to the vertebral body, the bone screw including an open concave head, a connecting element coupled to the bone screw, a joint element coupled around a middle cylindrical portion of the connecting element, an elongated bar element coupled to the upper spherical portion of the connecting element, and a pin adapted to fit inside the elongated bar element and a slot of the connecting element. The connecting element may include an upper spherical portion including a first diameter, a middle cylindrical portion including a second diameter less than the first diameter, and a lower spherical portion having a plurality of outwardly expandable legs adapted to lock into the open concave head of the bone screw.

### BACKGROUND

1. Technical Field

The embodiments herein generally relate to spinal fixation assemblies, and, more particularly, to a dynamic bone screw system for stabilizing a vertebral body.

2. Description of the Related Art

A spinal fixation device is a rigid or semi-rigid mechanical support system, which is surgically implanted into a vertebral column to obtain stabilization of spinal fractures, correction of spinal deformities, or treatment of degenerative spinal disease. The implanted fixation device may include rods, plates, and/or screws to provide support to vertebrae. Bone screws are one part of spinal fixation systems that allow mobility of the patient while treating damaged bone. The screws may be used to reclaim functionality lost due to osteoporotic fractures, traumatic injuries, or disc herniations.

Clinical experience indicates that a more rigid spinal stabilization system increases the risk of complications such as mechanical failure, device-related osteoporosis, and accelerated degeneration at adjoining levels. To avoid these complications and concurrently obtain adequate immobilization, it is important to stabilize the affected lumbar region while preserving the natural anatomy of the spine. Control of abnormal motions and more physiologic load transmissions may relieve pain and prevent adjacent segment degeneration. Thus, an ideal spinal fixation system should preferably provide hard immobilization as well as preservation of motion.

Traditional spinal fixation systems and bone screw assemblies tend to lack either translation for all directions or have a limitation of rotation. In those systems that provide for rotation, the center of rotation is typically not controlled. Also, there is generally a lack of limitation of the damping ability, which may lead to damage of the vertebrae during natural motion. Accordingly, there remains a need for a new spinal stabilization system to restore motion in a patient's back in a controlled manner while permitting natural motion with flexibility.

### SUMMARY

In view of the foregoing, an embodiment herein provides a dynamic bone screw system that includes a bone screw adapted to connect to a vertebral body, the bone screw including an open concave head, a connecting element coupled to the bone screw, a joint element coupled around a middle cylindrical portion of the connecting element, an elongated bar element coupled to an upper spherical portion of the connecting element, and a pin adapted to fit inside the elongated bar element and a slot of the connecting element.

The connecting element includes an upper spherical portion, a middle cylindrical portion, and a lower spherical portion. The upper spherical portion includes a first diameter, the middle cylindrical portion includes a second diameter less than the first diameter, and the lower spherical portion includes a dynamic third diameter capable of changing size. The lower spherical portion further includes a plurality of outwardly expandable legs adapted to lock into the open concave head of the bone screw. A plurality of channels in the lower spherical portion may separate the plurality of outwardly expandable legs. The slot is configured through an entire height of the upper spherical portion, the middle cylindrical portion, and the lower spherical portion. The insertion of the pin in the slot may cause each leg to outwardly expand. The connecting element may be adapted to rotate with respect to the bone screw. The elongated bar element may be adapted to rotate with respect to the connecting element and the pin. The elongated bar element may include an attachment head which may further include an aperture adapted to allow passage of the pin and a cavity connected to the aperture to engage the upper spherical portion of the connecting element and to allow passage of the pin. The joint element may be adapted to control a degree of rotation of the connecting element.

In another aspect, an apparatus for dynamically stabilizing a vertebral body includes a bone screw to connect to the vertebral body, a connecting element connected to the bone screw, a slot through an entire height of an upper spherical portion, a middle cylindrical portion, and a lower spherical portion, a joint element surrounding the middle cylindrical portion of the connecting element, an elongated bar element connected to the upper spherical portion of the connecting element, and a pin to fit inside the elongated bar element and the slot of the connecting element.

The bone screw includes an open concave head. The connecting element includes the upper spherical portion having a first diameter, the middle cylindrical portion having a second diameter less than the first diameter, and the lower spherical portion having a dynamic third diameter capable of changing size. The lower spherical portion further includes a plurality of outwardly expandable legs to lock into the open concave head of the bone screw. The connecting element may further include a plurality of channels in the lower spherical portion adapted to separate the plurality of outwardly expandable legs. The insertion of the pin in the slot may cause each leg to outwardly expand. The lower spherical portion is adapted to rotate with respect to the vertebral body and to translate the vertebral body in a first direction. The bar element is adapted to rotate with respect to the upper spherical portion and translate the vertebral body in a second direction. The connecting element may be adapted to rotate with respect to the bone screw.

The elongated bar element may include an attachment head which may further include an aperture to allow passage of the pin. The attachment head may further include a cavity connected to the aperture to engage the upper spherical portion of the connecting element and to allow passage of the pin. The elongated bar element may be adapted to rotate with respect to the connecting element and the pin. The joint element may be adapted to control a degree of rotation of the connecting element and to cushion an effect of translation of the vertebral body in the first direction and the second direction.

In yet another aspect, a method of performing a surgical procedure includes engaging a bone screw with a vertebral body, coupling a joint element around a connecting element, inserting a lower spherical portion of the connecting element in an open concave head of the bone screw, coupling an upper spherical portion of the connecting element to an elongated bar element, inserting a pin inside the elongated bar element and a slot of the connecting element, rotating the bar element with respect to the upper spherical portion of the connecting element to translate the vertebral body in a first direction, and rotating the lower spherical portion of the connecting element to translate the vertebral body in a second direction.

The connecting element includes the upper spherical portion having a first diameter, a middle cylindrical portion having a second diameter less than the first diameter, and the lower spherical portion having a dynamic third diameter capable of changing size. The lower spherical portion includes a plurality of outwardly expandable legs adapted to lock into the open concave head of the bone screw and a slot through an entire height of the upper spherical portion, the middle cylindrical portion, and the lower spherical portion. The connecting element may further include a plurality of channels in the lower spherical portion to separate the plurality of outwardly expandable legs. The insertion of the pin in the slot may cause each leg to outwardly expand.

The connecting element may be adapted to rotate with respect to the bone screw. The elongated bar element may include an attachment head which may further include an aperture to allow passage of the pin. The attachment head may further include a cavity connected to the aperture to engage the upper spherical portion of the connecting element and to allow passage of the pin. The elongated bar element may be adapted to rotate with respect to the connecting element and the pin. The joint element may be adapted to control a degree of rotation of the connecting element and to cushion an effect of translation of the vertebral body in the first direction and the second direction.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates an exploded perspective view of a dynamic screw system according to an embodiment herein;

FIGS. 2(A) and 2(B) illustrate assembled views of the dynamic screw system of FIG. 1, according to an embodiment herein;

FIGS. 3A through 3C illustrate a front view, a sectional view, and a top view, respectively, of the bone screw of the dynamic screw system of FIG. 1 according to an embodiment herein;

FIGS. 4A through 4D illustrate a front view, a sectional view, a perspective view, and a top view, respectively, of the connecting element of the dynamic screw system of FIG. 1 according to an embodiment herein;

FIGS. 5A through 5D illustrate a front view, a sectional view, a perspective view, and a top view, respectively, of the joint element of the dynamic screw system of FIG. 1 according to an embodiment herein;

FIGS. 6A through 6D illustrate a perspective view, a sectional view, a top view, and a side view, respectively, of the bar element of the dynamic screw system of FIG. 1 according to an embodiment herein;

FIGS. 7A through 7C illustrate a front view, a perspective view, and a bottom view respectively of the stationary element of the dynamic screw system of FIG. 1 according an embodiment herein; and

FIG. 8 is a process flow diagram that illustrates a method of performing a surgical procedure according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

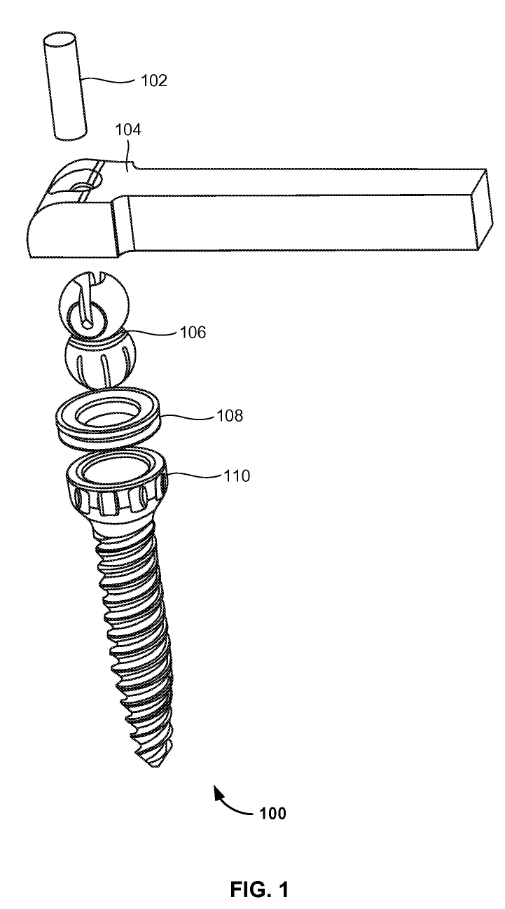

As mentioned, there remains a need for a new spinal stabilization system to restore motion in a patient's back in a controlled manner while permitting natural motion with flexibility. The embodiments herein achieve this by providing a dynamic bone screw system for insertion into a vertebral body, wherein the screw system includes a bar element, a bone screw adapted to connect to the vertebral body, a connecting element operatively connected to the bone screw, and a joint element coupled around the connecting element to mitigate an effect of a movement of the vertebral body. Referring now to the drawings, and more particularly to FIGS. 1 through 8, where similar reference characters denote corresponding features consistently throughout the figures, there are shown preferred embodiments.

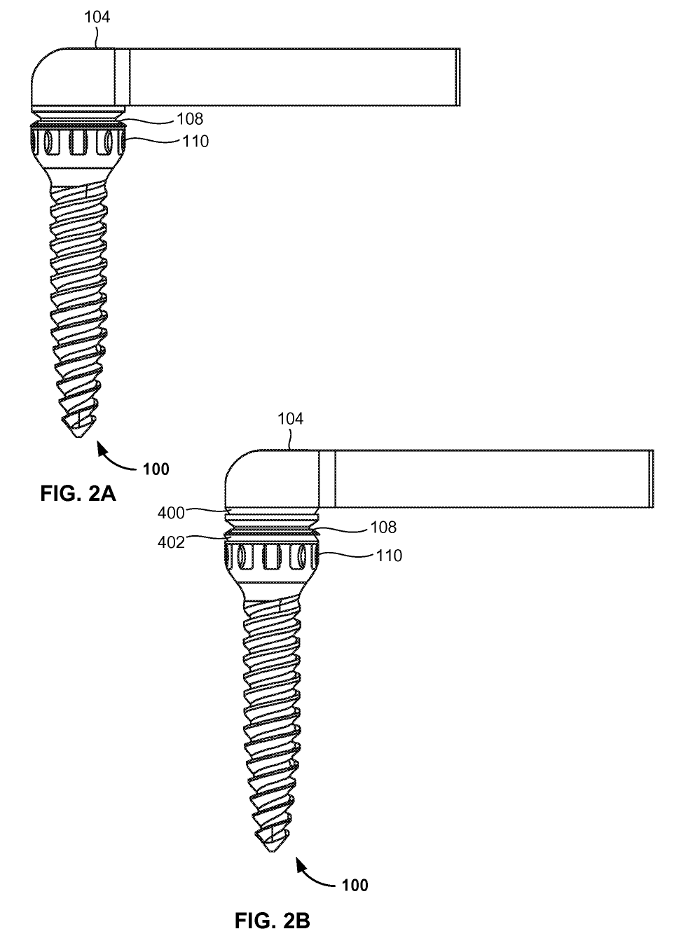

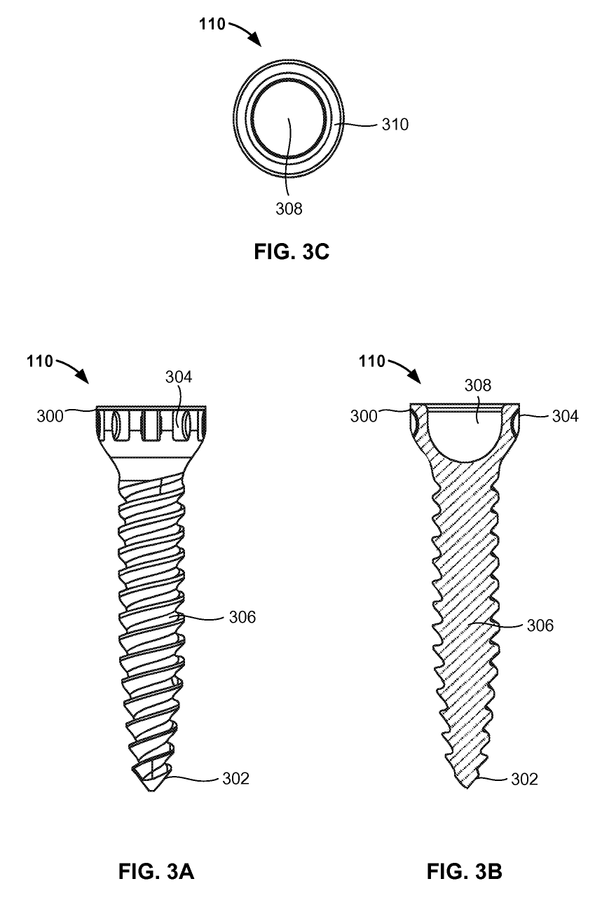

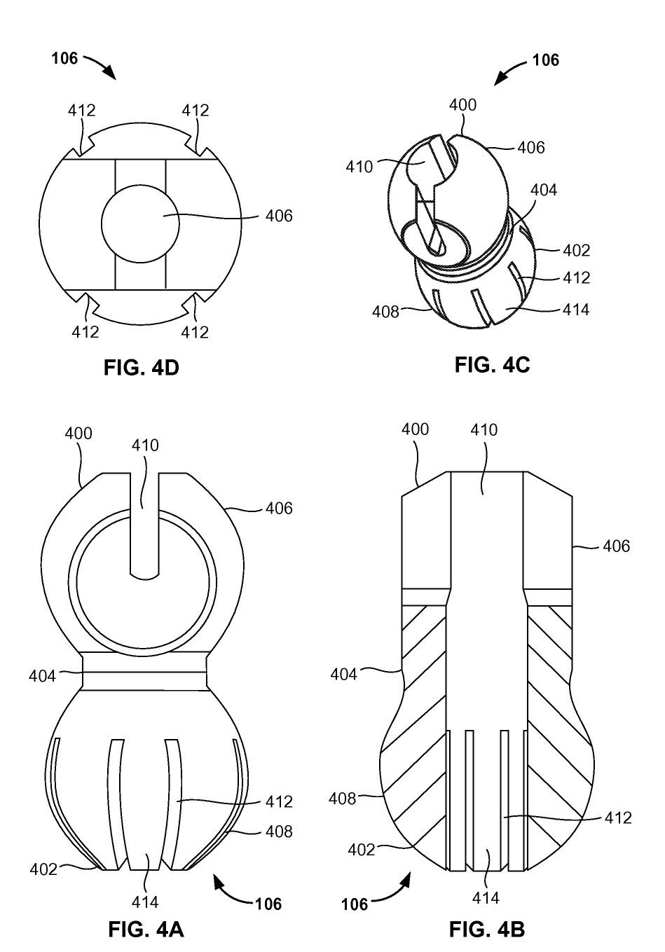

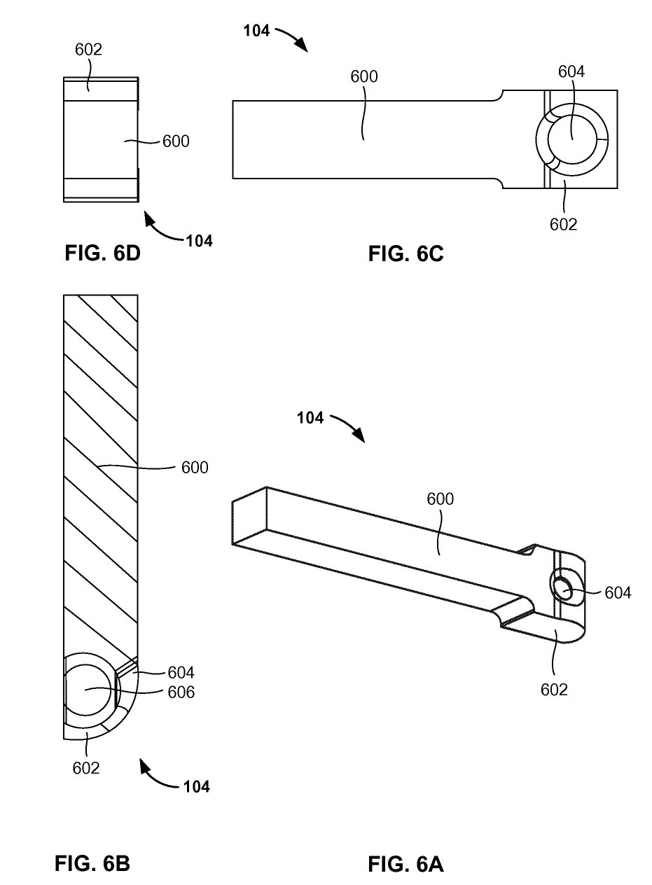

FIG. 1 illustrates an exploded perspective view of a dynamic screw system 100 having a stationary element 102, a bar element 104, a connecting element 106, a joint element 108, and a bone screw 110 according to an embodiment herein. FIGS. 2(A) and 2(B) illustrate an assembled view of the dynamic screw system 100 of FIG. 1. With reference to FIGS. 1 through 2(B), the stationary element 102 may be embodied as a pin and is dimensioned and configured to fit into the bar element 104. The bar element 104, which is an elongated cross bar, at its bottom portion (e.g., the cavity 606 of FIG. 6B) may be coupled to the connecting element 106 by the stationary element 102. The connecting element 106 is dimensioned and configured to fit into the bone screw 110 (e.g., through the lower spherical portion 402 of FIGS. 4A through 4C and the open concave head 300 and the cavity 308 of FIGS. 3A through 3C). The joint element 108 may be positioned around the connecting element 106 (e.g., in the middle cylindrical portion 404 between the upper spherical portion 400 and the lower spherical portion 402 of the connecting element 106 of FIGS. 4A through 4D).

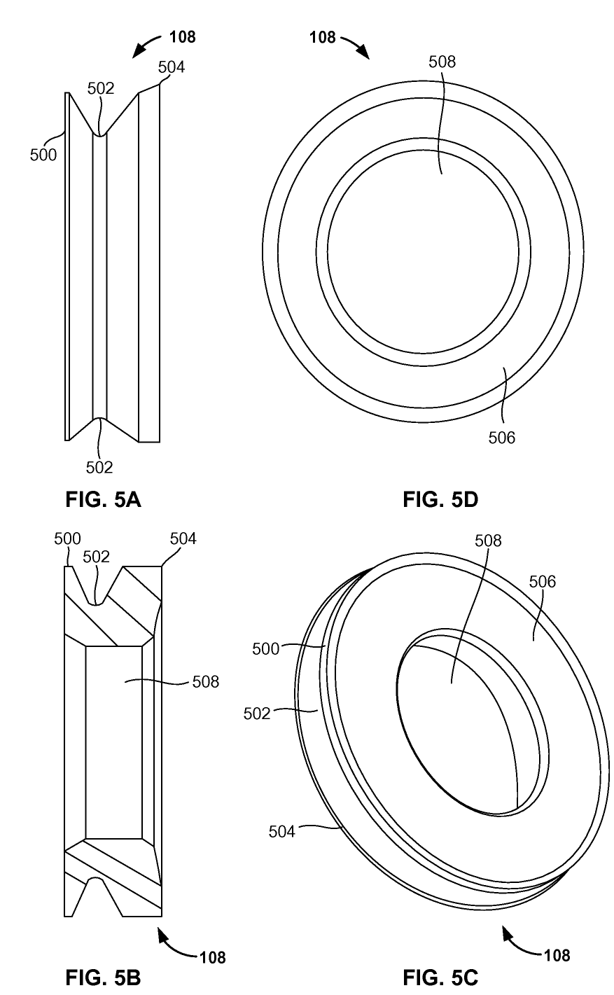

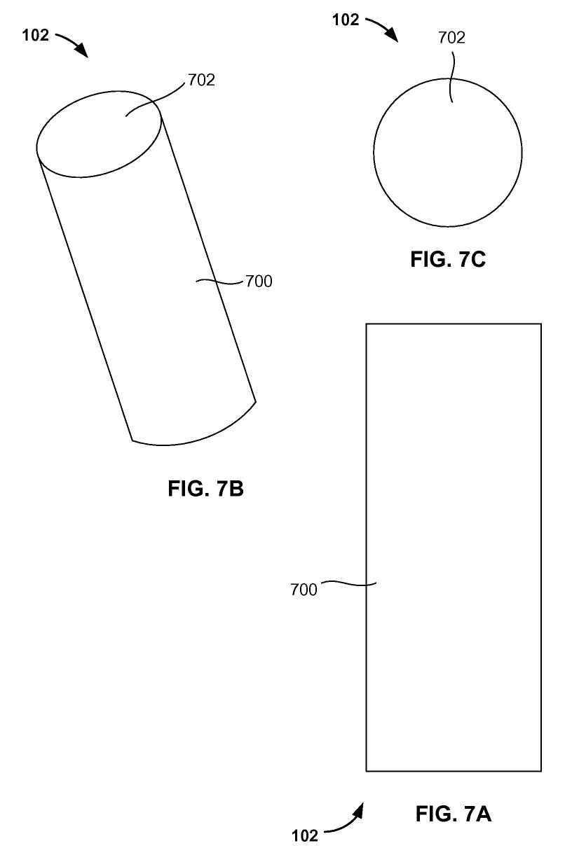

The stationary element 102 may pass through the bar element 104 (e.g., the cylindrical portion 700 and the end 702 of FIGS. 7B and 7C through the aperture 604 and cavity 606 of FIGS. 6A through 6C) and may be received by the connecting element 106 (e.g., through the “U” shaped slot 410 of FIG. 4A through 4D). The stationary element 102 may prevent the connecting element 106 from decoupling from the bar element 104. The connecting element 106 may be configured to allow the bar element 104 (e.g., through the upper spherical portion 400 of FIGS. 4A through 4C and the head 602 and the cavity 606 of FIGS. 6A through 6C) to rotate with respect to an upper center 406 of the upper spherical portion 400 of the connecting element 106. The connecting element 106 may be operatively connected to the joint element 108 (e.g., through the narrowed cylindrical middle portion 404 of FIGS. 4A through 4C and inner hollow portion 508 of FIGS. 5B through 5D). The joint element 108 may be coupled to the bone screw 110 to mitigate an effect (e.g., may provide a damping or cushioning) of a movement of the vertebral body (e.g., bending or stretching of the vertebral body).

The connecting element 106 is fitted into the bone screw 110 (e.g., through the lower spherical portion 402 of FIGS. 4A through 4C and the open concave head 300 of FIGS. 3A and 3B). The bone screw 110 is operatively connected to a vertebral body (not shown) (e.g., by the threaded screw portion 306 and the pointed end 302 of FIGS. 3A and 3B). The attachment of the connecting element 106 to the bone screw 110 and then to the vertebral body allows the vertebral body to rotate with respect to the lower center 408 of the lower spherical portion 402 of the connecting element 106 (e.g., through the middle cylindrical portion 404 of FIGS. 4A through 4C) to translate the vertebral body in a first direction (e.g., in a superior direction). The bar element 104 may be configured to rotate with respect to the upper center 406 of the upper spherical portion 400 of the connecting element 106 (e.g., through the middle cylindrical portion 404 of FIGS. 4A through 4C) and translate the vertebral body in a second direction (e.g., in an inferior direction). Double rotations create sliding motions in one plane. The first rotation on the upper spherical portion 400 provides one directional rotation; however, the lower spherical portion 402 can lead to the second rotation, which can be a reversed rotation with respect to the first rotation occurred by the upper spherical portion 400. Thus, these two rotations create either a double pendulum motion or a sliding/translating motion of the vertebral body. The direction of the vertebral body translation will occur in the superior/inferior direction as well as the posterior/anterior direction.

FIGS. 3A through 3C illustrate a front view, a sectional view, and a top view, respectively, of the bone screw 110 of the dynamic screw system 100 of FIG. 1 according to an embodiment herein. FIG. 3A is the front view of the bone screw 110 of the dynamic screw system 100 which may have an open concave head 300 with grooves 304. The open concave head 300 may have a threaded portion 306 which extends from the bottom end of the open concave head 300 to a pointed end 302. FIG. 3B illustrates the sectional view having the open concave head 300, the pointed end 302, the grooves 304, and the threaded portion 306. The open concave head 300 may have an internal cavity 308. FIG. 3C is the top view which shows the top of the bone screw 110 having the internal cavity 308 and the external annular lip 310. The bone screw 110 may include the threaded portion 306 and the pointed end 302 to anchor into vertebra (not shown). The open concave head 300 with the internal cavity 308 is dimensioned and configured to accommodate the connecting element 106 (e.g., through the lower spherical portion 402 of FIGS. 4A through 4C). The grooves 304 permit the gripping of an inserter device, such as a screwdriver, to the bone screw 110. The annular lip 310 may fix the cushion joint element 108 (e.g., through the outer ring 506 of FIGS. 5C and 5D).

FIGS. 4A through 4D illustrate a front view, a sectional view, a perspective view, and a top view, respectively, of the connecting element 106 of the dynamic screw system 100 of FIG. 1 according to an embodiment herein. FIG. 4A is the front view of the connecting element 106 which illustrates the upper spherical portion 400 having an upper center 406, the lower spherical portion 402 having a lower center 408, and the middle cylindrical portion 404. The upper spherical portion 400 may comprise a first diameter. The middle cylindrical portion 404 may have a second diameter which is less than the first diameter of the upper spherical portion 400. The lower spherical portion 402 may have a dynamic third diameter capable of changing size due to the expandable feature provided by the legs 414. The “U” shaped slot 410 is present in the upper spherical portion 400 while the lower portion 402 may have some channels 412 defining expandable legs 414. The channels 412 at the lower spherical portion 402 separate the expandable legs 414. FIG. 4B is the sectional view showing the upper spherical portion 400 with the upper center 406, the lower spherical portion 402 with the lower center 408, the middle cylindrical portion 404, the slot 410, the channels 412 and the legs 414. The slot 410 may be configured through an entire height of the upper spherical portion 400, the middle cylindrical portion 404, and the lower spherical portion 402. FIG. 4C illustrates a three-dimensional perspective view of the connective element 106 having the upper spherical portion 400 with the upper center 406, the lower spherical portion 402 with the lower center 408, the middle cylindrical portion 404, the slot 410, the channels 412, and the expandable legs 412. FIG. 4D is the top view which shows the generally circular configuration of the slot 410 (to match the circumferential configuration of the stationary element 102).

The upper spherical portion 400 fits into the bar element 104 (e.g., in the cavity 606 of the attachment head 602 of FIGS. 6A through 6C) while the lower spherical portion 402 may be fitted into the bone screw 110 (e.g., through the open concave head 300 and the cavity 308 of FIGS. 3A through 3C). Additionally, the middle cylindrical portion 404 is configured to accommodate the joint element 108 (e.g., through the inner hollow portion 508 of FIGS. 5C and 5D) also to allow the joint element 108 to pass through to the lower spherical portion 402. The “U” shaped slot 410 positioned at the upper spherical portion 400 extends through the entire height of the connecting element 106 and is dimensioned and configured to accommodate the stationary element 102 (e.g., the cylinder 700 and the end 702 of FIGS. 7B through 7C). When the stationary element 102 is inserted in the slot 410 and reaches the area of the lower portion 402 of the connecting element 106, each of the expandable legs 414 of the connecting element 106 expand outwardly into the internal cavity 308 of the open concave head 300 of the bone screw 110, thereby locking the connecting element 106 to the bone screw 110. However, the curved configuration of the lower portion 402 of the connecting element 106 also facilitates the rotation of the connecting element 106 (with the attached bar element 104) with respect to the stationary element 102. This arrangement of the connecting element 106 allows the bar element 104 and the bone screw 110 to rotate with respect to the middle cylindrical portion 404 of the connecting element 106. These two rotations of the connecting element 106 allow the vertebrae to translate into the first and second directions (e.g., superior and inferior directions).

FIGS. 5A through 5D illustrate a front view, a sectional view, a perspective view, and a top view respectively of the joint element 108 of the dynamic screw system 100 of FIG. 1 according to an embodiment herein. The joint element 108, which is positioned above the bone screw 110 (as shown in FIGS. 1 through 2(B)) is configured as a ring-like structure comprising an upper conical portion 500, a middle cylindrical portion 502, a lower conical portion 504, an outer ring 506, and an inner hollow portion 508 to allow the connecting element 106 (of FIGS. 4A through 4D) to be inserted through the joint element 108 and attach to the bone screw 110. The upper conical portion 500 of the joint element 108 is adapted to allow the connecting element 106 (e.g., through the upper spherical portion 400 of FIGS. 4A through 4C) to rest thereon. Additionally, the middle cylindrical portion 502 of the joint element 108 is adapted to accommodate the connecting element 106 within the bone screw 110 (e.g. through the cavity 308 of FIGS. 3A and 3B and the lower spherical portion 402 of FIGS. 4A through 4C) to cushion an effect of translation (e.g., of the vertebral body towards or away from the bar element 104). Furthermore, the lower conical portion 504 of the joint element 108 is appropriately contoured to match the configuration of the connecting element 106 (e.g., of the lower spherical portion 402 of FIGS. 4A through 4C). Generally, the outer ring 506 controls the degree of rotation of the connecting element 106 once the connecting element 106 is fit through the joint element 108 and seated in the open concave head 300 of the bone screw 110. The inner hollow portion 508 allows the connecting element 106 to pass through it (e.g., through the middle cylindrical portion 404 of FIGS. 4A though 4C). Additionally, the joint element 108 may comprise flexible polymer material, silicon, urethane, or metallic materials, for example. Preferably, the joint element 108 cushions the effect of the translation of the vertebral body in the first and second directions (e.g., in the superior and inferior directions) by absorbing contraction and expansion forces during the movement of the spine.

FIGS. 6A through 6D illustrate a perspective view, a sectional view, a top view, and a side view respectively of the bar element 104 of the dynamic screw system 100 of FIG. 1 according to an embodiment herein. The bar element 104 comprises a generally rectangular plate 600 connected to a broadened attachment head 602 with an aperture 604 connecting to a cavity 606. The rectangular plate 600 may allow the bar element 104 to rotate with respect to the center of the connecting element 106 (e.g., middle cylindrical portion 404 of FIGS. 4A through 4C). Furthermore, the attachment head 602 and the cavity 606 may be configured to receive the upper spherical portion 400 of connecting element 106. The aperture 604 and cavity 606 may be configured to allow the passage of the stationary element 102 (of FIGS. 7B and 7C) therein.

The other end of the bar element 104 connects to either a regular pedicle fixation system (not shown), any type of fixation system (not shown), or another dynamic pedicle screw system (not shown). If the other end of the bar element 104 connects to a fixation system, the vertebral body connected to the bone screw 110 can have a constrained six degrees of freedom of motion with respect to the vertebral body connected to the fixation system. However, if the other end of the bar element 104 connects to another dynamic screw system 100, then the vertebral body connected to the bone screw 110 can have a double six degrees of freedom of motion with respect to the vertebral body connected to the dynamic screw system 100.

FIGS. 7A through 7C illustrate a front view, a perspective view, and a bottom view respectively of the stationary element 102 of the dynamic screw system 100 of FIG. 1 according an embodiment herein. Generally, the stationary element 102 is configured as a cylindrical structure, although other configurations are possible. The stationary element 102 generally comprises cylinder 700 with a plurality of opposed ends 702. With respect to FIGS. 1 through 7C, the cylinder 700 of the stationary element 102 is appropriately shaped to first allow the stationary element 102 to easily pass through the aperture 604 and cavity 606 of bar element 104 then to be received through the slot 410 of the upper spherical portion 400 of the connecting element 106. Then, the stationary element 102 may be extended into the lower spherical portion 402 of the connecting element 106, thereby engaging connecting element 106 into the open concave head 300 of the bone screw 110 (e.g., by engaging and extending the legs 414 of FIGS. 4A through 4C outward). This arrangement of the stationary element 102 also prevents the connecting element 106 from decoupling from the bar element 104.

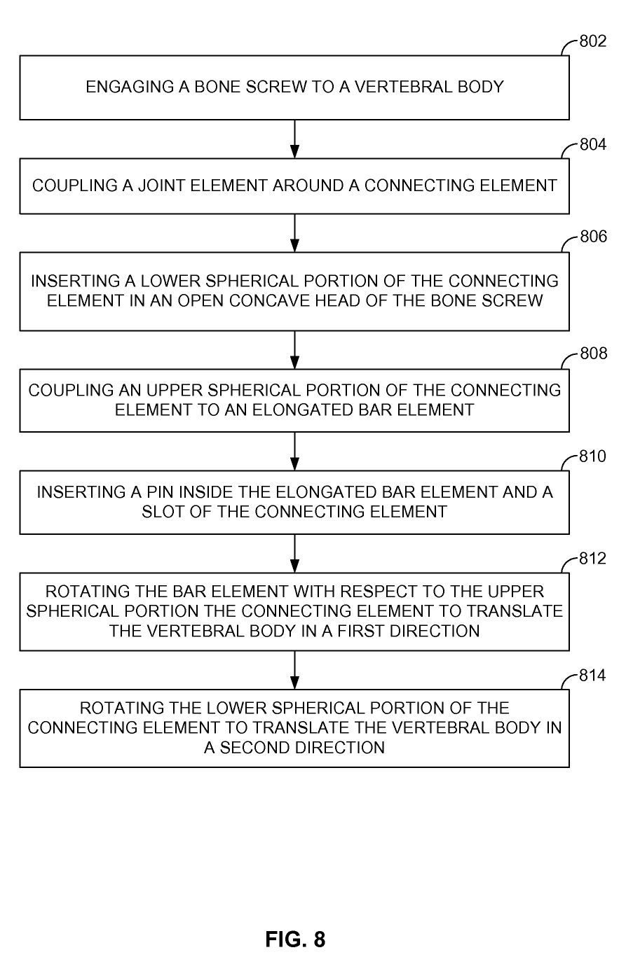

FIG. 8, with reference to FIGS. 1 through 7C, is a process flow diagram that illustrates a method of performing a surgical procedure according to an embodiment herein, wherein the method comprises engaging (802) the bone screw 110 of a dynamic screw system 100 with a vertebral body (not shown), coupling (804) the joint element 108 around the connecting element 106, inserting (806) the lower spherical portion 402 of the connecting element 106 in the open concave head 300 of the bone screw 110, coupling (808) the upper spherical portion 400 of the connecting element 106 to the elongated bar element 104, inserting (810) the stationary element (pin) 102 inside the elongated bar element 104 and the slot 410 of the connecting element 104, rotating (812) the bar element 104 with respect to the upper spherical portion 400 of the connecting element 106 to translate the vertebral body in a first direction, and rotating (814) the lower spherical portion 402 of the connecting element 106 to translate the vertebral body in a second direction.

In step (802), the bone screw 110 of the dynamic screw system 100 is engaged with a vertebral body. The bone screw 110 may be anchored into the vertebral body (e.g., through the threaded portion 306 and the pointed end 302 as shown in FIGS. 3A and 3B). In step (804), the joint element 108 is coupled around the middle cylindrical portion 404 of the connecting element 106). In step (806), the lower spherical portion 402 of the connecting element 106 may be inserted in the open concave head 300 of the bone screw 110. In step (808), the upper spherical portion 400 of the connecting element 106 is coupled to the elongated bar element 104 (e.g., through the cavity 606 of the attachment head 602 of FIGS. 6A through 6C). In step (810), the stationary element (pin) 102 is inserted inside the elongated bar element 104 (e.g., through the aperture 604 and the cavity 606 of FIGS. 6A through 6C) and the slot 410 of the connecting element 104 (e.g., through the cylinder 700 and end 702 of FIGS. 7A through 7C). In step (812), the bar element 104 is rotated with respect to the upper spherical portion 400 of the connecting element 106 (e.g., through the cavity 606 of FIGS. 6A through 6C) to translate the vertebral body in a first direction. In step (814), the lower spherical portion 402 of the connecting element 106 is rotated to translate the vertebral body in a second direction.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A dynamic screw system comprising:
a bone screw adapted to connect to a vertebral body, wherein said bone screw comprises an open concave head;
a connecting element coupled to said bone screw, wherein said connecting element comprises:
an upper spherical portion comprising a first diameter;
a middle cylindrical portion comprising a second diameter less than said first diameter;
a lower spherical portion having a plurality of outwardly expandable legs adapted to lock into said open concave head of said bone screw, wherein said lower spherical portion comprises a dynamic third diameter capable of changing size; and
a slot configured through an entire height of said upper spherical portion, said middle cylindrical portion, and said lower spherical portion;
a joint element coupled around said middle cylindrical portion of said connecting element;
an elongated bar element coupled to said upper spherical portion of said connecting element; and
a pin adapted to fit inside said elongated bar element and said slot of said connecting element.

2. The dynamic screw system of claim 1, wherein said connecting element is adapted to rotate with respect to said bone screw, and wherein said elongated bar element is adapted to rotate with respect to said connecting element.

3. The dynamic screw system of claim 1, wherein said elongated bar element is adapted to rotate with respect to said pin.

4. The dynamic screw system of claim 2, wherein said joint element is adapted to control a degree of rotation of said connecting element.

5. The dynamic screw system of claim 1, wherein said connecting element further comprises a plurality of channels in said lower spherical portion and adapted to separate said plurality of outwardly expandable legs, wherein insertion of said pin in said slot causes each leg to outwardly expand.

6. The dynamic screw system of claim 1, wherein said bar element comprises an attachment head comprising:
an aperture adapted to allow passage of said pin; and
a cavity connected to said aperture and adapted to engage said upper spherical portion of said connecting element, and to allow passage of said pin.

7. An apparatus for dynamically stabilizing a vertebral body, said apparatus comprising:
a bone screw adapted to connect to a vertebral body, wherein said bone screw comprises an open concave head;
a connecting element coupled to said bone screw, wherein said connecting element comprises:
an upper spherical portion comprising a first diameter;
a middle cylindrical portion comprising a second diameter less than said first diameter;
a lower spherical portion having a plurality of outwardly expandable legs adapted to lock into said open concave head of said bone screw, wherein said lower spherical portion comprises a dynamic third diameter capable of changing size, wherein said lower spherical portion is adapted to rotate with respect to said vertebral body and to translate said vertebral body in a first direction; and
a slot configured through an entire height of said upper spherical portion, said middle cylindrical portion, and said lower spherical portion;
a joint element coupled around said middle cylindrical portion of said connecting element;
an elongated bar element coupled to said upper spherical portion of said connecting element, wherein said elongated bar element is adapted to rotate with respect to said upper spherical portion and translate said vertebral body in a second direction; and
a pin adapted to fit inside said elongated bar element and said slot of said connecting element.

8. The apparatus of claim 7, wherein said connecting element is adapted to rotate with respect to said bone screw, and wherein said elongated bar element is adapted to rotate with respect to said connecting element.

9. The apparatus of claim 7, wherein said elongated bar element is adapted to rotate with respect to said pin.

10. The apparatus of claim 8, wherein said joint element is adapted to control a degree of rotation of said connecting element.

11. The apparatus of claim 7, wherein said connecting element further comprises a plurality of channels in said lower spherical portion and adapted to separate said plurality of outwardly expandable legs, wherein insertion of said pin in said slot causes each leg to outwardly expand.

12. The apparatus of claim 7, wherein said bar element comprises an attachment head comprising:
an aperture adapted to allow passage of said pin; and
a cavity connected to said aperture and adapted to engage said upper spherical portion of said connecting element, and to allow passage of said pin.

13. The apparatus of claim 7, wherein said joint element is adapted to cushion an effect of translation of said vertebral body in said first direction and said second direction.

14. A method of performing a surgical procedure, said method comprising:
engaging a bone screw to a vertebral body, wherein said bone screw comprises an open concave head;
coupling a joint element around a connecting element, wherein said connecting element comprises:
an upper spherical portion comprising a first diameter;
a middle cylindrical portion comprising a second diameter less than said first diameter;
a lower spherical portion having a plurality of outwardly expandable legs adapted to lock into said open concave head of said bone screw, wherein said lower spherical portion comprises a dynamic third diameter capable of changing size; and
a slot configured through an entire height of said upper spherical portion, said middle cylindrical portion, and said lower spherical portion,
wherein said joint element is coupled around said middle cylindrical portion of said connecting element;
inserting said lower spherical portion of said connecting element in said open concave head of said bone screw;
coupling said upper spherical portion of said connecting element to an elongated bar element; and
inserting a pin inside said elongated bar element and said slot of said connecting element;
rotating said bar element with respect to said upper spherical portion of said connecting element to translate said vertebral body in a first direction; and
rotating said lower spherical portion of said connecting element to translate said vertebral body in a second direction.

15. The method of claim 14, wherein said connecting element is adapted to rotate with respect to said bone screw, and wherein said elongated bar element is adapted to rotate with respect to said connecting element.

16. The method of claim 14, wherein said elongated bar element is adapted to rotate with respect to said pin.

17. The method of claim 15, wherein said joint element is adapted to control a degree of rotation of said connecting element.

18. The method of claim 14, wherein said connecting element further comprises a plurality of channels in said lower spherical portion and adapted to separate said plurality of outwardly expandable legs, wherein insertion of said pin in said slot causes each leg to outwardly expand.

19. The method of claim 14, wherein said bar element comprises an attachment head comprising:
an aperture adapted to allow passage of said pin; and
a cavity connected to said aperture and adapted to engage said upper spherical portion of said connecting element, and to allow passage of said pin.

20. The method of claim 14, wherein said joint element is adapted to cushion an effect of translation of said vertebral body in said first direction and said second direction.
